(19) United States
(12) Patent Application Publication
(10) Pub. No.: US 2013/0337053 A1
(43) Pub. Date: Dec. 19, 2013

(54) NOVEL FORMULATIONS AND METHODS

(71) Applicants: Leo RUBIN, Suffern, NY (US); Shaker MOUSA, Wynantskill, NY (US); Edward MATTES, Tuxedo Park, NY (US)

(72) Inventors: Leo RUBIN, Suffern, NY (US); Shaker MOUSA, Wynantskill, NY (US); Edward MATTES, Tuxedo Park, NY (US)

(21) Appl. No.: 13/918,539

(22) Filed: Jun. 14, 2013

Related U.S. Application Data
(60) Provisional application No. 61/660,405, filed on Jun. 15, 2012.

(51) Int. Cl. A61K 38/22; A61K 45/06; A61K 31/197; A61K 9/48

(52) U.S. Cl. CPC A61K 38/2278; A61K 9/48; A61K 45/06; A61K 31/197
USPC 424/451; 514/13.1

(57) ABSTRACT

A formulation comprising T3/VIP nanoparticles, wherein the T3/VIP nanoparticle comprises both T3 and VIP encapsulated or immobilized on a bioabsorbable polymer. The invention further provides for methods of making a formulation comprising a T3/VIP nanoparticle. The invention further provides for methods of treatment utilizing said T3/VIP nanoparticle.

### RELATED APPLICATIONS

The application claims the benefit of U.S. Provisional Application 61/660,405 filed Jun. 15, 2012 the contents of each of which are incorporated herein by reference.

### TECHNICAL FIELD

The field relates to nanoparticles comprising vasoactive intestinal peptide (VIP) and triiodothyronine (T3), and to their use in treatment of pulmonary hypertension, vascular and neurological disorders, autoimmune disorders and cardiac conditions, for example cardiac arrest and acute heart failure.

### BACKGROUND OF THE INVENTION

Pulmonary hypertension (PH or PHT) is an increase in blood pressure in the pulmonary artery, pulmonary vein, and/or pulmonary capillaries. It is a very serious condition, potentially leading to shortness of breath, dizziness, fainting, decreased exercise tolerance, heart failure, pulmonary edema, and death. It can be one of five different groups, classified by the World Health Organization as follows:

WHO Group I—Pulmonary arterial hypertension (PAH)
a. Idiopathic (IPAH)
b. Familial (FPAH)
c. Associated with other diseases (APAH): collagen vascular disease (e.g. scleroderma), congenital shunts between the systemic and pulmonary circulation, portal hypertension, HIV infection, drugs, toxins, or other diseases or disorder.
d. Associated with venous or capillary disease
   Pulmonary arterial hypertension involves the vasoconstriction or tightening of blood vessels connected to and within the lungs. This makes it harder for the heart to pump blood through the lungs, much as it is harder to make water flow through a narrow pipe as opposed to a wide one. Over time, the affected blood vessels become both stiffer and thicker, in a process known as fibrosis. This further increases the blood pressure within the lungs and impairs their blood flow. In addition, the increased workload of the heart causes thickening and enlargement of the right ventricle, making the heart less able to pump blood through the lungs, causing right heart failure. As the blood flowing through the lungs decreases, the left side of the heart receives less blood. This blood may also carry less oxygen than normal. Therefore it becomes more and more difficult for the left side of the heart to pump to supply sufficient oxygen to the rest of the body, especially during physical activity.
   WHO Group II—Pulmonary hypertension associated with left heart disease
a. Atrial or ventricular disease
b. Valvular disease (e.g. mitral stenosis)
   In pulmonary venous hypertension (WHO Group II) there may not be any obstruction to blood flow in the lungs. Instead, the left heart fails to pump blood efficiently out of the heart into the body, leading to pooling of blood in veins leading from the lungs to the left heart (congestive heart failure or CHF). This causes pulmonary edema and pleural effusions. The fluid build-up and damage to the lungs may also lead to hypoxia and consequent vasoconstriction of the pulmonary arteries, so that the pathology may come to resemble that of Group I or III.
   WHO Group III—Pulmonary hypertension associated with lung diseases and/or hypoxemia
a. Chronic obstructive pulmonary disease (COPD), interstitial lung disease (ILD)
b. Sleep-disordered breathing, alveolar hypoventilation
c. Chronic exposure to high altitude
d. Developmental lung abnormalities
   In hypoxic pulmonary hypertension (WHO Group III), the low levels of oxygen may cause vasoconstriction or tightening of pulmonary arteries. This leads to a similar pathophysiology as pulmonary arterial hypertension.
   WHO Group IV—Pulmonary hypertension due to chronic thrombotic and/or embolic disease
a. Pulmonary embolism in the proximal or distal pulmonary arteries
b. Embolization of other matter, such as tumor cells or parasites
   In chronic thromboembolic pulmonary hypertension (WHO Group IV), the blood vessels are blocked or narrowed with blood clots. Again, this leads to a similar pathophysiology as pulmonary arterial hypertension.

### WHO Group V—Miscellaneous

Treatment of pulmonary hypertension has proven very difficult. Antihypertensive drugs that work by dilating the peripheral arteries are frequently ineffective on the pulmonary vasculature. For example, calcium channel blockers are effective in only about 5% of patients with IPAH. Left ventricular function can often be improved by the use of diuretics, beta blockers, ACE inhibitors, etc., or by repair/replacement of the mitral valve or aortic valve. Where there is pulmonary arterial hypertension, treatment is more challenging, and may include lifestyle changes, digoxin, diuretics, oral anticoagulants, and oxygen therapy are conventional, but not highly effective. Newer drugs targeting the pulmonary arteries, include endothelin receptor antagonists (e.g., bosentan, sitaxentan, ambrisentan), phosphodiesterase type 5 inhibitors (e.g., sildenafil, tadalafil), prostacyclin derivatives (e.g., epoprostenol, treprostenil, iloprost, beroprost), and soluble guanylate cyclase (sGC) activators (e.g., cinaciguat and riociguat). Surgical approaches to PAH include atrial septostomy to create a communication between the right and left atria, thereby relieving pressure on the right side of the heart, but at the cost of lower oxygen levels in blood (hypoxia); lung transplantation; and pulmonary thromboendarterectomy (PTE) to remove large clots along with the lining of the pulmonary artery.

Heart failure and acute myocardial infarction are common and serious conditions frequently associated with thrombosis and/or plaque build-up in the coronary arteries. Many patients who have suffered one or more myocardial infarctions subsequently require treatment for congestive heart failure (CHF). The left heart fails while the pumping function of the right heart remains adequate, because the latter has only about 20% of the workload of the former. This leads to an increase in blood volume congested to the lungs, resulting in pulmonary congestion, build up of edema, and congestion of internal organs including the stomach and intestines. Increased fluid in the stomach and intestines reduce their ability to absorb drugs prescribed for treatment of CHF, particularly diuretics. The congestion is often accompanied by a worsening of myocardial function, with consequent drop in blood pressure and reduced renal perfusion, which only further aggravates the congestive situation.

Cardiac arrest refers to a state where the heart of the patient has stopped beating effectively and is no longer functioning to pump blood around the body. It is often caused by myocardial infarction. If treated promptly, cardiac arrest may sometimes reversed by cardiopulmonary resuscitation (CPR) and defibrillation. Drugs to treat cardiac arrest include epinephrine, which stimulates the heart muscle and also augments pressure in the aorta, which drives coronary perfusion. Whether epinephrine significantly improves overall survival is controversial, however, because while it may improve the chances for resuscitation, it may also cause arrhythmias and strain on the heart which increase the risk of problems in the post-resuscitation phase.

Other forms of acute cardiac insufficiency include acute heart failure and cardiogenic shock. Acute heart failure is a critical condition that is commonly seen in patients with chronic heart disease.

Vasoactive intestinal peptide (VIP) is a peptide hormone containing 28 amino acid residues, produced in many areas of the human body including the gut, pancreas and suprachiasmatic nuclei of the hypothalamus in the brain. In humans, the vasoactive intestinal peptide is encoded by the VIP gene. Various synthetic forms of VIP or VIP from other mammalian sources are known. VIP causes vasodilatation, lowers arterial blood pressure, stimulates myocardial contractility, increases glycogenolysis and relaxes the smooth muscle of trachea, stomach and gall bladder. VIP is a potent dilator of the pulmonary and coronary arteries, and has great potential to reduce pulmonary arterial hypertension and at the same time enhance cardiac function. VIP is also known to dilate the cardiac arteries and to enhance cardiac function. VIP is therefore useful to treat acute myocardial infarction and to treat heart failure resulting from myocardial infarction. It thus has potential to help patients having conditions such as pulmonary hypertension, cardiac insufficiency, heart failure, and acute myocardial infarction. To date, however, it has not been used as a therapeutic because it has a half-life (T_{1/2}) in the blood of less than two minutes.

There is thus an unmet need for improved treatments for pulmonary hypertension, particularly pulmonary arterial hypertension, for cardiac insufficiency due to partial or complete blockage of coronary arteries and/or damage due to myocardial infarction, for example acute or congestive heart failure and acute myocardial infarction. There is moreover a need for a means of sustaining levels of VIP for longer periods of time, e.g. to treat such conditions.

Triiodothyronine, also known as T3, is a thyroid hormone. Thyroid-stimulating hormone (TSH) activates the production of thyroxine (T4) and T3. T4 is converted to T3 by deiodination. T3 affects a variety of body processes, including body temperature, growth, and heart rate. T3 has important effects on cardiac tissue. Thyroid hormones, notably T3, modulate ventricular function via genomic and non-genomic mechanisms. Cardiac stress events (cardiac arrest, myocardial infarction, etc.) are associated with steep reductions in serum T3 levels. Post resuscitation T3 level correlates highly with survival rate. T3 additionally has cardiostimulatory properties: it increases the cardiac output by decreasing systemic vascular resistance and force of contraction. Overall, there is reason to believe that early bolus T3 injection could increase chances of resuscitating cardiac arrest victims, and that elevating T3 serum concentration could increase prospects of survival to hospital discharge.

T3 is not currently approved for this indication, however, and current formulations of T3 are not well suited for this purpose. Triostat® requires refrigeration, making it somewhat impractical for emergency use. Also, the concentration is low for what is needed to treat cardiac arrest. T3-albumin formulation have been described but are difficult to make, and like Triostat®, have poor stability and are poorly suited for quick administration in an emergency setting.

### SUMMARY OF THE INVENTION

The invention provides T3/VIP nanoparticles, wherein the nanoparticle comprises T3 and VIP, wherein the T3 and VIP are encapsulated or immobilized by a bioabsorbable polymer having any of the following characteristics
a. Wherein the polymer comprises chitosan.
b. Wherein the polymer comprises poly(lactic-co-glycolic acid) (PLGA) or polylactic acid (PLA), e.g., PLGA having 50/50 co-polymerization of D,L-lactic acid and glycolic acid.
c. Wherein the polymer comprises chitosan crosslinked using glutaraldehyde.
d. Wherein the polymer comprises chitosan linked to bile acids.
e. Wherein the polymer comprises chitosan linked to PLGA, e.g., using glutaraldehyde as crosslinker.
f. Any of the foregoing wherein the nanoparticle comprises acetic acid and/or an acetic acid analogue, e.g., Dicholoroacetic acid (“DCA”).
g. Any of the foregoing wherein the nanoparticles have an average diameter of 50-1000 nm, e.g., 100-500 nm or 50-250 nm.
h. Any of the foregoing wherein the nanoparticles have a zeta potential of 10-100 mV.
i. Any of the foregoing wherein the nanoparticle comprises a second pharmacologically active ingredient.

In one embodiment, the nanoparticle comprises T3 and VIP, and a bioabsorbable polymer, wherein the nanoparticle further comprises T3 on the outside of the nanoparticle and VIP on the inside of the nanoparticle. It is contemplated that in this embodiment that T3 would have non-genomic applications. It is further contemplated that in this embodiment that VIP would be protected from degradation as well as an increased half-life. It is further contemplated that in this embodiment that VIP would have a sustained release, and a targeted delivery, e.g. to the vascular bed. In one further embodiment, T3 is covalently linked to the outside of the nanoparticle.

In one embodiment, the T3 is covalently linked to the bioabsorbable polymer, for example via the hydroxy on the phenyl moiety, and VIP is encapsulated or immobilized in the bioabsorbable polymer. Such compositions can be formed using, e.g., activated T3 which is activated at the phenolic hydroxy with a suitable linker and protected at the amino moiety. The amino-protected T3 is then linked to the nanoparticle, for example via the phenolic hydroxy, e.g. by using an activated linker group, for example a moiety capable of coupling to an amine group on the bioabsorbable polymer, for example the amino moieties on chitosan.

In one embodiment, the invention provides a nanoparticle comprising T3 and VIP, wherein the T3 is an activated T3 which is substituted on the phenolic hydroxy group with an epoxide moiety of formula [CH2-O—CH]—[CH2]ₙ- and which is amino protected. For example, the invention provides a T3/VIP nanoparticle compound wherein T3 is formula 1:

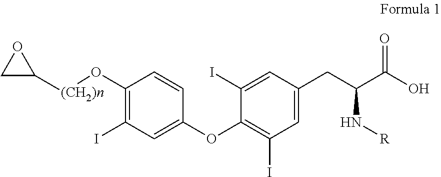

wherein n is an integer selected from 1 through 5, and R is an amino protecting group, e.g., butoxycarbonyl (BOC).

In one embodiment, the T3/VIP nanoparticle can comprise a T3 which may be activated, for example using an epoxyalkyl of formula [CH2-O—CH]—[CH2]ₙ-X wherein n=1-5 and X is halogen, e.g. bromine, e.g. according to a synthesis as shown in FIG. 10. The resulting compound is then, if necessary, selectively deprotected to release the carboxy moiety, for example,

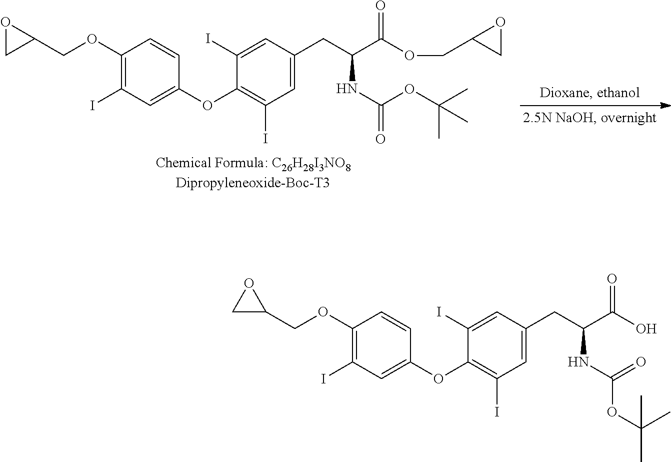 [SMILES: CC(C)(C)OC(=O)N[C@@H](Cc1cc(I)c(Oc2ccc(OCC3CO3)c(I)c2)c(I)c1)C(=O)OCC1CO1 ; CC(C)(C)OC(=O)N[C@@H](Cc1cc(I)c(Oc2ccc(OCC3CO3)c(I)c2)c(I)c1)C(=O)O]

to provide T3 which is activated at the phenolic hydroxy (here, with propylene oxide) and amino-protected (here, with BOC).

The activated T3 may be attached to the bioabsorbable polymer, for example, T3 having an epoxy linker moiety and an amino-protecting group is reacted with a bioabsorbable polymer having amino groups, then deprotected to provide a nanoparticle covalently linked to T3, e.g., as shown in FIG. 7. This reaction may be carried out in the presence of a stabilizer, such as polyvinyl alcohol, e.g. PVA 1% w/v, in an appropriate solvent, for example dimethylsulfoxide, e.g. DMSO (0.1% v/v) and acetic acid (0.1% v/v), which solvents are removed afterwards by dialysis. The number of T3 moieties attached to the nanoparticle may vary based on the reaction conditions and amount of reactant used, but if these conditions are kept constant, the distribution of variation will be low. Typically, the nanoparticle will comprise 20-200 T3 moieties, e.g., about 50 per nanoparticle. The amount of T3 in a batch can be assayed, e.g., as described below, by separating the nanoparticles by filtration or centrifugation, weighing, degrading the T3/VIP nanoparticle in strong base, and measuring by HPLC.

The overall scheme for activation of T3, and subsequent immobilization on a T3/VIP nanoparticle may be shown, for example, in FIG. 8.

In one example, the T3/VIP nanoparticles comprise T3, VIP, and the following components:

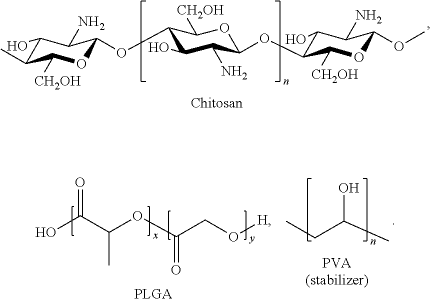

In one example, the nanoparticles have these components in approximately the following amounts:

| | Approx Amount | |
|---|---|---|
| Components of | (% w/w) in the | |
| the formulation | nanoformulation | Role in the formulation |
| Chitosan | 50-70%, e.g. 60% | Component of the nanocarrier |
| PLGA | 20-30%, e.g. 25% | Component of the nanocarrier |
| T3 and VIP | 10-20%, e.g. 15% | Active ingredients (chemically |
| | | conjugated to the nanoparticles) |

The contents of the nanoparticles are confirmed using, e.g. HPLC and LC/MS. The nanoparticle formulations may be sterilized using conventional means, e.g., filtration, gamma radiation.

In one further aspect, the invention provides T3/VIP nanoparticles, wherein the nanoparticle comprises T3 and VIP, wherein the T3 and VIP are encapsulated or immobilized by a bioabsorbable polymer (e.g., having any of the characteristics of foregoing list a.-i.), wherein the bioabsorbable polymer is chitosan, for example, and wherein the chitosan has any of the following characteristics:
j.) The chitosan is derived from fungus, e.g. mushroom or mold;
k.) The chitosan is derived from fungus, wherein the fungus is an Aspergillus, e.g., Aspergillus niger;
l.) The chitosan is derived from mushroom, wherein the mushroom is an Agaricus, e.g., Agaricus bisporus;
m.) Any of the foregoing wherein the chitosan has a molecular weight range of about Mv 30,000-220,000;
n.) Any of the foregoing wherein the chitosan a molecular weight range of about Mv 30,000-60,000;
o.) Any of the foregoing wherein the chitosan has a range of apparent viscosity (e.g. at 1% solution in 1% acetic acid) of about <20 mPa·s to 90 (+/−30 mPa·s); e.g. <20 mPa·s, 40 (+/−20 mPa·s), 55 (+/−25 mPa·s), 90 (+/−30 mPa·s);
p.) Any of the foregoing wherein the chitosan has a degree of acetylation (% mol) in a range of about 10%-40%;
q.) Any of the foregoing wherein the chitosan has a degree of acetylation (% mol) in a range of about e.g, 10%-20%, 15%-25%, 20%-30%, or 30%-40%.
r.) Any of the foregoing wherein the T3 is encapsulated within the chitosan, and wherein there is a greater ratio of chitosan present in the nanoparticle relative to the amount of PLGA, e.g. a relative ratio amount of 80/20, chitosan to PLGA, (e.g., % w/w 80/20, chitosan to PLGA)

The above measurements may be carried out by any means known in the art. For example, it is contemplated that the viscosity of chitosan solutions may be measured at room temperature using a Brookfield type digital viscometer, e.g., DV-11+Pro. In another example, it is contemplated that the viscosity may be measured using a Ubbelohde type viscometer. In such an example, it is contemplated that the viscometer could be connected to a visco-clock to record the time of the passing solution.

In one aspect, the present invention provides for T3/VIP nanoparticles wherein the nanoparticle comprises T3, VIP, and chitosan, e.g., chitosan having any of the characteristics of foregoing list a.)-r.), and PLGA, wherein the relative ratio of chitosan to PLGA may be altered to adjust the release of the active ingredient, e.g. T3 or VIP. Without being bound by theory, it is believed that chitosan is hydrophilic. Therefore, where the active ingredient may possibly be hydrophobic (e.g. T3) the addition of more chitosan relative to PLGA may result in a nanoparticle wherein the active ingredient is quickly released upon application or administration, e.g., a relative ratio amount of 80/20, (e.g., % w/w 80/20, chitosan to PLGA) chitosan to PLGA, or a relative ratio amount of 90/10 (e.g., % w/w 90/10, chitosan to PLGA) chitosan to PLGA. Without being bound by theory, where the active ingredient is more hydrophobic, the addition of more PLGA, relative to the amount of chitosan, may result in a nanoparticle wherein the active ingredient is more slowly released, e.g., a relative ratio of 20/80 chitosan to PLGA (e.g., % w/w 20/80, chitosan to PLGA), or 10/90 chitosan to PLGA (e.g., % w/w 10/90, chitosan to PLGA)

In one embodiment, the invention provides a method for treating an acute cardiac condition, e.g. cardiac arrest, cardiac arrhymia, or cardiac insufficiency, comprising administering an effective amount of a T3/VIP-nanoparticle (e.g., a nanoparticle of any of the foregoing lists a.)-r.)) to a patient in need thereof, wherein the T3/VIP-nanoparticle comprises a bioabsorbable polymer, for example as described above.

In one embodiment, the invention provides a method for treating a cardiac condition, e.g., right and left sided heart failure, right sided heart failure with pulmonary hypertension, comprising administering an effective amount of a T3/VIP-nanoparticle (e.g., a nanoparticle of any of the foregoing lists a.)-r.)) to a patient in need thereof, wherein the T3/VIP nanoparticle comprises a biosbsorbable polymer, for example as described herein.

In another embodiment, the invention provides for a method of treating pulmonary hypertension, comprising administering an effective amount of a T3/VIP nanoparticle formulation (e.g., a nanoparticle of any of the foregoing lists a.)-r.)) to a patient in need thereof. The present invention contemplates that the T3/VIP nanoparticle may be used to treat either primary or secondary pulmonary hypertension. It is further contemplated by the present invention that a T3/VIP nanoparticle formulation may be administered in conjunction with: endothelin receptor antagonists (e.g., bosentan, sitaxentan, ambrisentan), phophodiesterase type 5 inhibitors (e.g., sildenafil, tadalafil), prostacylin derivatives (e.g., epoprostenol, treporostenil, iloprost, beroprost), and soluble guanylate cyclase (sGC) activators (e.g., cinaciguat and riociguat).

In yet another example, the present invention contemplates the use of a T3/VIP nanoparticulate formulation (e.g., a nanoparticle of any of the foregoing lists a.)-r.)) to treat a patient in need thereof. It is contemplated by the present invention that a T3/VIP nanoparticulate formulation may be used to treat cardiac insufficiency, e.g., heart failure, right-sided heart failure, angina, congestive heart failure or acute myocardial infarction.

In another embodiment, the present invention provides for a method of treating intraabdominal pressure and/or intraabdominal hypertension. It is contemplated by the present invention that the T3/VIP nanoparticulate formulation (e.g., a nanoparticle of any of the foregoing lists a.)-r.)) could used to treat cardiorenal failure which could result from the occurrence of intraabdominal pressure and/or intraabdominal hypertension.

In another embodiment, the present invention provides for a method of treating autoimmune disorders, e.g. scleroderma, lupus, as they relate to pulmonary hypertension and cardiac conditions, e.g. heart failure, comprising administering an effective amount of a T3/VIP nanoparticle formulation to a patient in need thereof.

In another embodiment, the present invention contemplates the use of a T3/VIP nanoparticulate formulation (e.g., a nanoparticle of any of the foregoing lists a.)-r.)) to treat organs used or intended to be used for transplantation from a donor to a recipient. The present invention contemplates that the organs are donor organs. The present invention contemplates that the donor and recipient are the same species. The present invention also contemplates that the donor and recipient are of different species. The present invention provides for a method of treating organs, comprising administering an effective amount of a T3/VIP nanoparticle to a donor organ, e.g. kidney, heart, lung, heart/lung, liver, either prior to and/or contemporaneously with transplant. It is contemplated that the administration of a T3/VIP nanoparticle would enhance perfusion and/or reduce the risk of rejection, e.g. the risk of chronic rejection. It is contemplated that administration of a composition comprising a T3/VIP nanoparticle includes continuous infusion of said T3/VIP nanoparticle prior to implantation. It is also contemplated that the composition comprising the T3/VIP nanoparticle can be administered to a recipient after transplantation.

In one example of the method, the T3/VIP-nanoparticle administered comprises a chitosan-PLGA nanoparticles encapsulating and/or immobilizing T3 and VIP.

In another example, the administered T3/VIP-nanoparticle comprises chitosan nanoparticles encapsulating T3 and/or VIP with glutaraldehyde as a cross linker. Other cross-linkers may be used. In yet another example, the administered T3/VIP-nanoparticle comprises chitosan-PLGA nanoparticles encapsulating T3 and/or VIP. Such examples of T3/VIP nanoparticles may utilize a process that includes gelation/conjugation of preformed biodegradable polymers.

In yet another example, the T3/VIP-nanoparticle administered includes chitosan-PLGA nanoparticles immobilizing T3. Alternatively, the administered T3/VIP-nanoparticles comprise chitosan-PLGA nanoparticles immobilizing T3 and/or VIP as well as chitosan-PLGA nanoparticles encapsulating T3 and/or VIP.

In another example, the T3/VIP-nanoparticle comprises T3 and/or VIP covalently linked to chitosan or chitosan-PLGA nanoparticles.

Nanoparticle production is generally described in the Applicant's own publications: US 20110142947 A1, and WO 2011/159899, the contents of each of which are incorporated herein by reference in their entireties. In one embodiment, T3 nanoparticles produced by these methods disclosed herein, and the Applicants own publications, could be further contacted with VIP in order to produce a T3/VIP combination nanoparticle. Conversely, it also contemplated that a VIP nanoparticles produced by these methods could be further contacted with T3 in order to produce a T3/VIP combination nanoparticle. In one embodiment, T3 and VIP could be applied contemporaneously. It is contemplated that chitosan derived from fungus (e.g., having any of the characteristics of foregoing j.)-r.)) could be utilized in any of the nanoparticle production methods described or disclosed herein.

### BRIEF DESCRIPTION OF THE FIGURES

FIG. 1 depicts a flow chart diagram for the synthesis of encapsulating VIP in the T3/VIP chitosan nanoparticles.

FIG. 2A, depict flow charts for the synthesis of chitosan PLGA-nanoparticles. FIG. 2B depicts a PLGA-chitosan T3/VIP nanoparticle wherein VIP is encapsulated. (T3 not pictured)

FIG. 3 depicts a schematic flowchart of various methods of synthesizing T3/VIP nanoparticles that either encapsulate or immobilize T3 and VIP.

FIGS. 4A and 4B show an example of a flowchart of encapsulating T3 in PLGA-Chitosan T3/VIP nanoparticles. (VIP not pictured)

FIG. 5 shows one embodiment of the synthesis of amino-protected T3 that may be used in a T3/VIP nanoparticle.

FIG. 6 shows one method in which T3 may thus be activated, for example using an epoxyalkyl of formula [CH2-O—CH]—[CH2]ₙ-X wherein n=1-5 and X is halogen, e.g. bromine.

FIG. 7 shows one example of activated T3 attached to the bioabsorbable polymer of a T3/VIP nanoparticle, for example, T3 having an epoxy linker moiety and an amino-protecting group is reacted with a bioabsorbable polymer having amino groups, then deprotected to provide a nanoparticle covalently linked to T3.

FIG. 8 shows one example of T3 attached to the bioabsorbable polymer of the T3/VIP nanoparticle. (VIP not pictured)

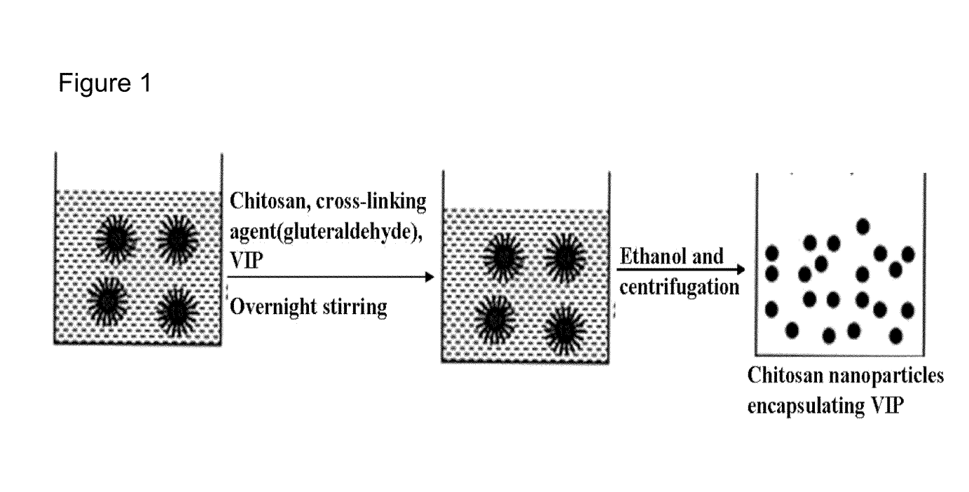

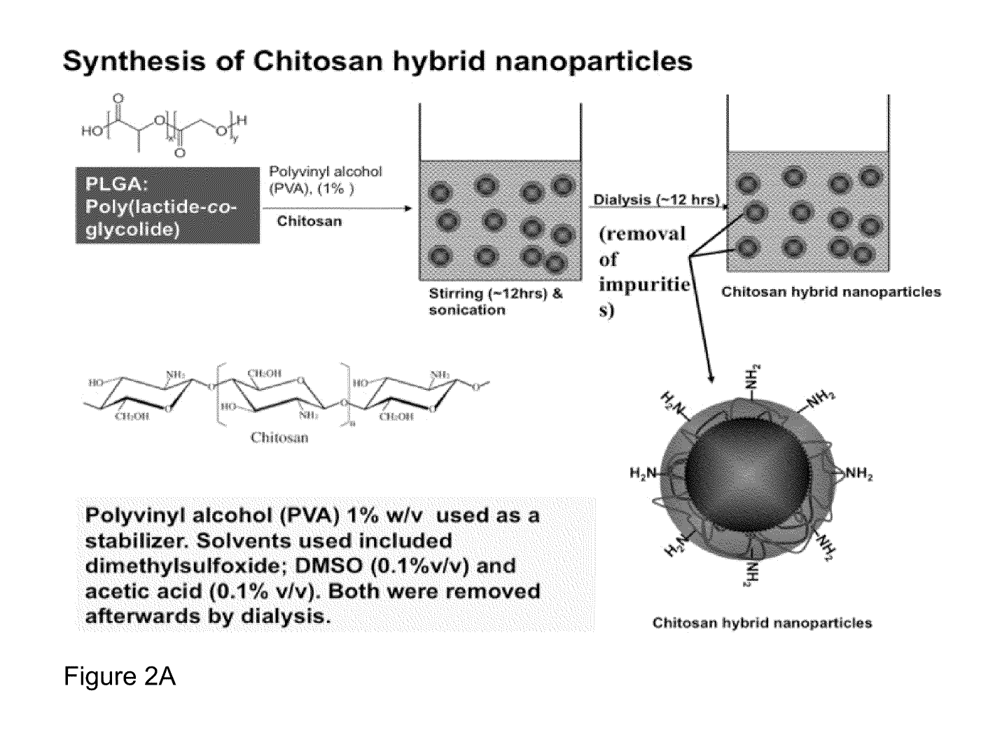

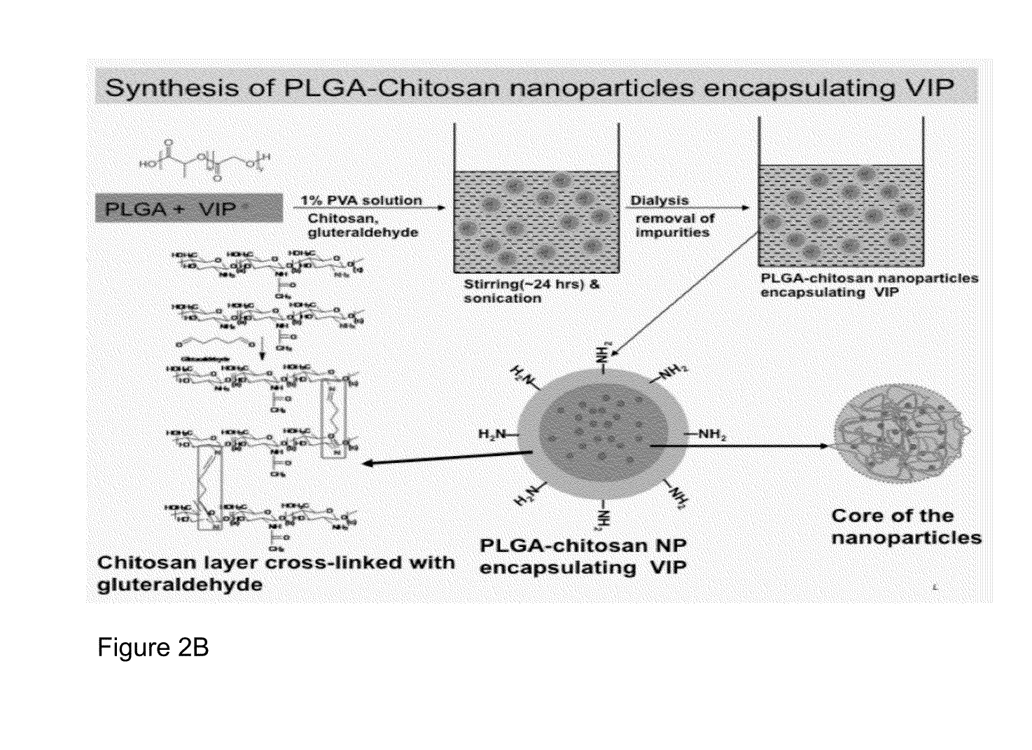

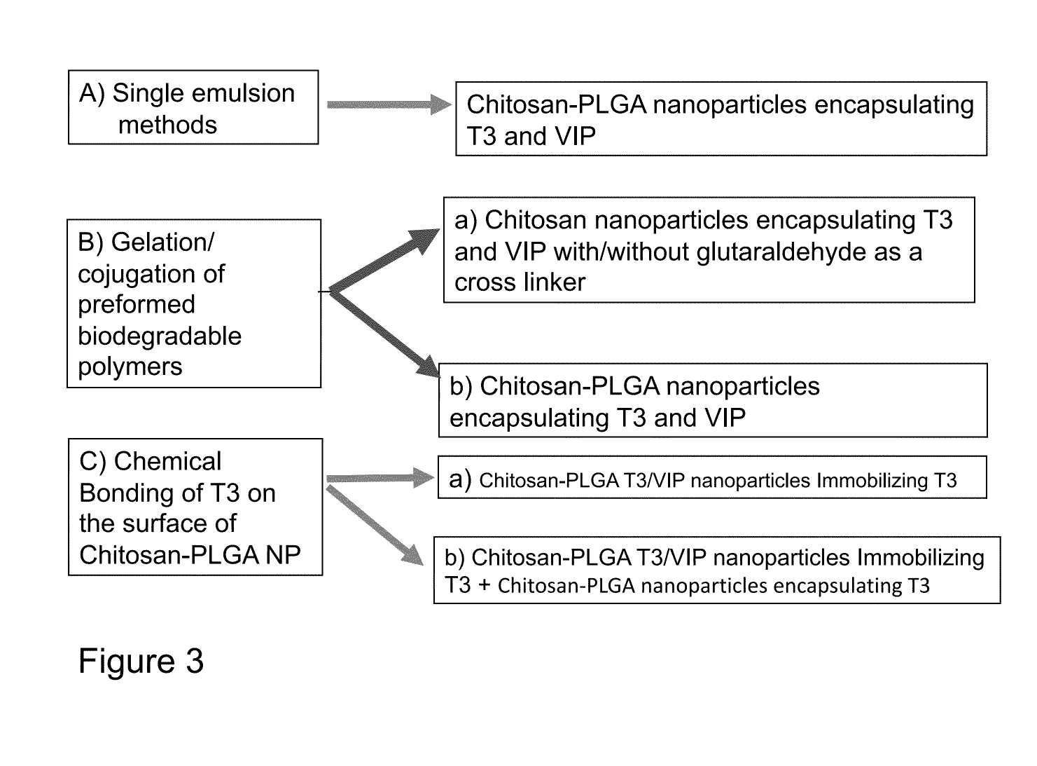

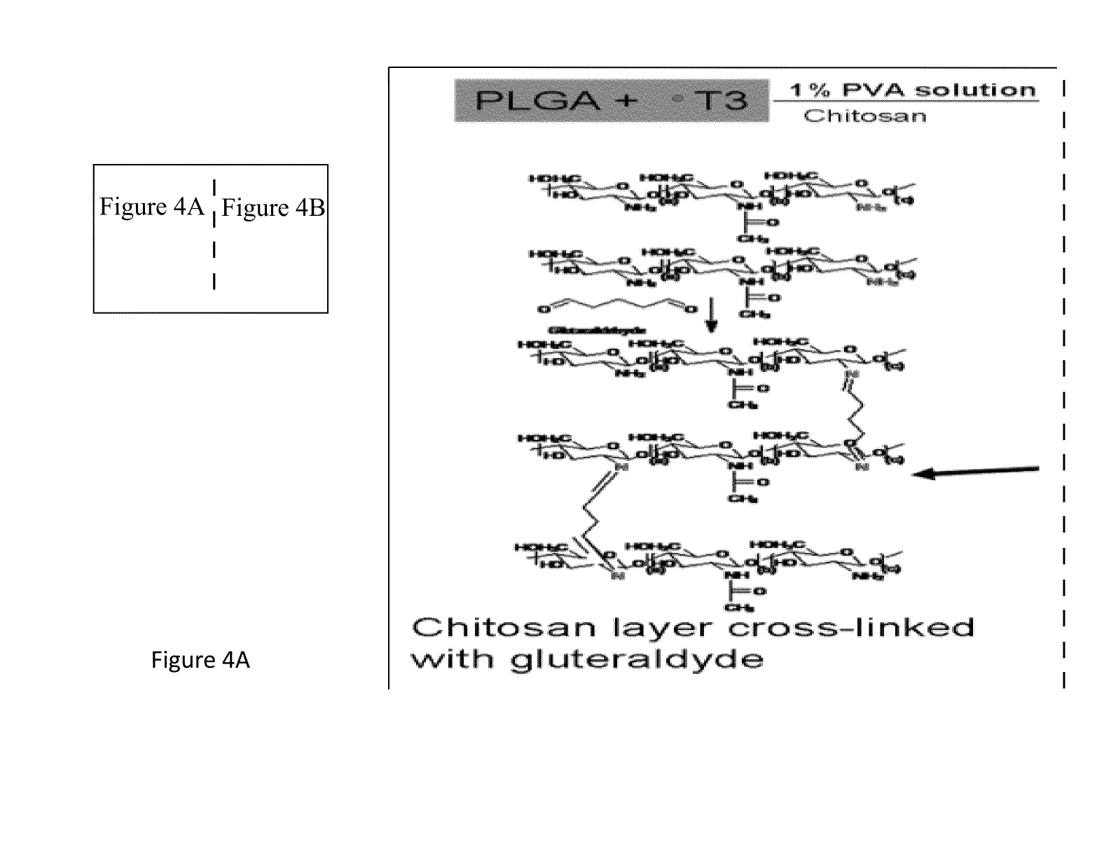

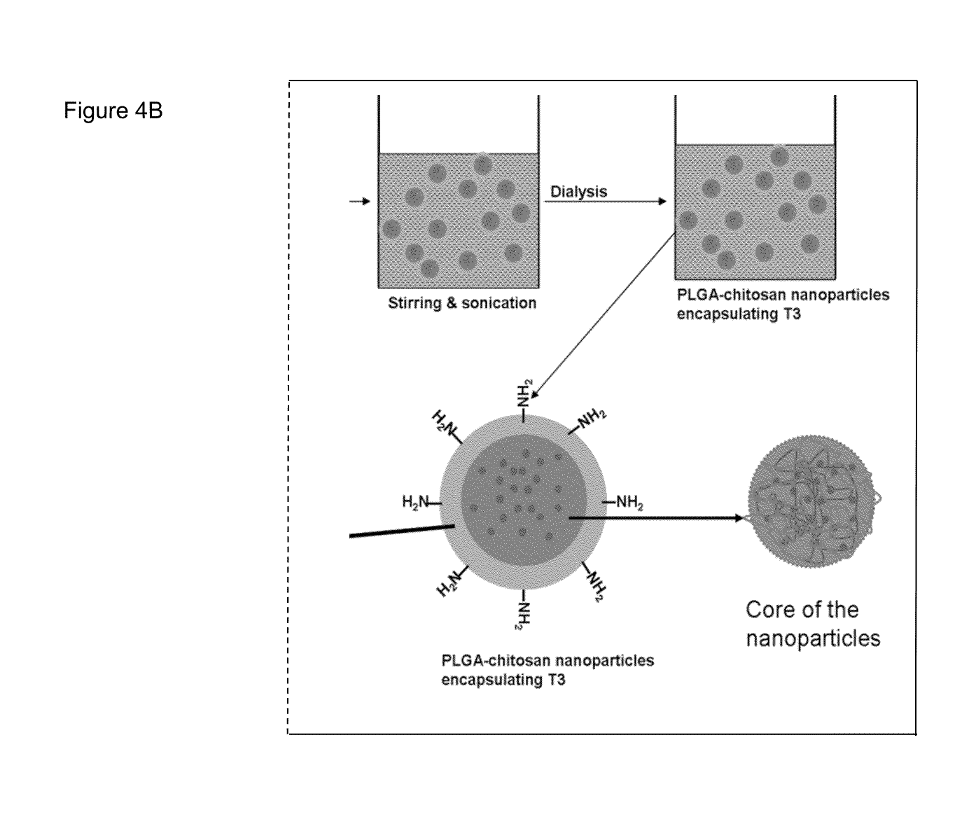

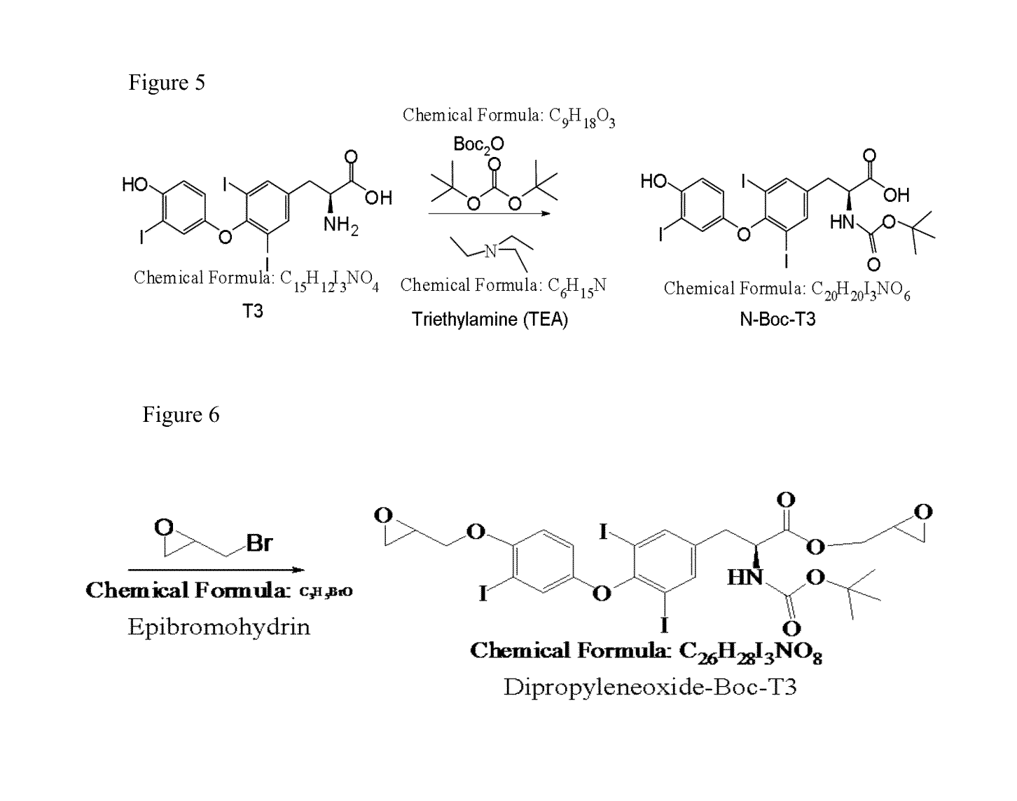

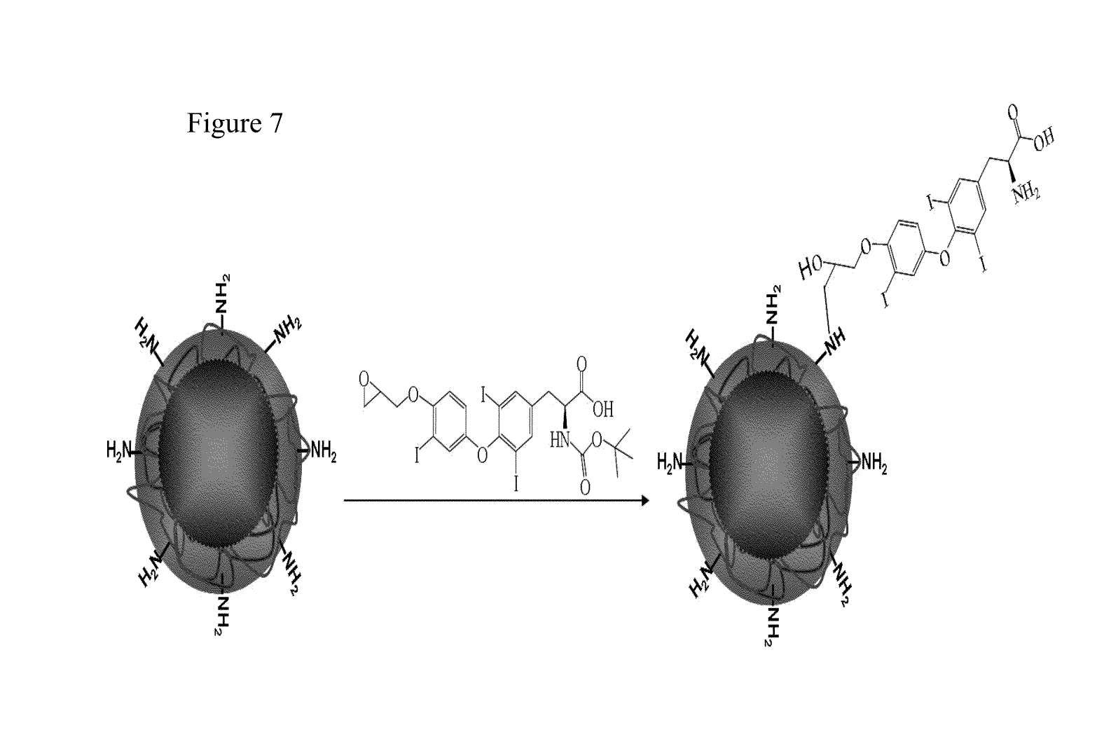

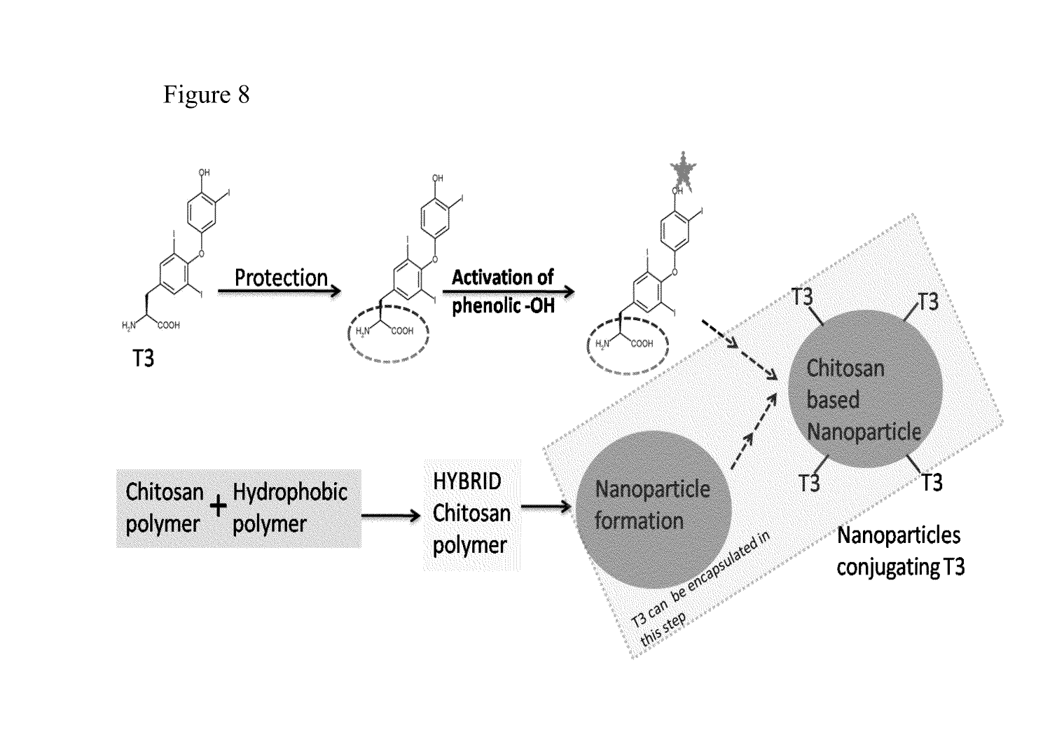

### DETAILED DESCRIPTION

The invention provides T3/VIP nanoparticles, wherein the nanoparticle comprises T3 and VIP (e.g., of any of the foregoing a.)-r)), and wherein T3 and VIP are either encapsulated or immobilized by a bioabsorbable polymer (e.g., chitosan). VIP, as used herein includes any peptide or peptide analogue having VIP activity, e.g., capable of binding VPAC₁ or VPAC₂, esp. VPAC₁, e.g. selected from
s. Human VIP, e.g His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn-NH₂;
t. VIP from other mammals, e.g., porcine VIP;
u. Active fragments or derivatives, of human or other mammalian VIP, e.g. comprising at least residues 11-27 of VIP.
v. Human or other mammalian VIP precusor protein;
w. Analogues of VIP, e.g. (Ala(2,8,9,11,19,22,24,25,27,28)VIP, (Ala(2,8,9,11,19,24-28))VIP, (Ala(2,8,9,16,19,24))VIP, (Ala(2,8,9,16,19,24,25))VIP, e.g., as disclosed in Igarashi H., et al. J Pharmacol Exp Ther. 2005 October; 315(1):370-81; Tyr(9), Dip(18)]VIP(1-28), e.g., as disclosed in Tams, et al, Mol. Pharmacol. 2000 November; 58(5):1035-41; R15, 20, 21, L17]-VIP-GRR as disclosed in Ohmori, et al. Life Sci. 2006 Jun. 6; 79(2):138-43; stearyl-norleucine-vasoactive intestinal peptide (stearyl-Nle17-VIP), as described in Gozes, et al, Endocrinology. 1994 May; 134(5):2121-5; Ro 24-9881 or Ro 25-1553, e.g., as described in O'Donnell et al, J Pharmacol Exp Ther. 1994 September; 270(3):1282-8; or analogues as disclosed in U.S. Pat. No. 7,094,755 and U.S. Pat. No. 4,835,252; other linear and cyclic VIP analoges as known in the art; the contents of the foregoing publication and patents being incorporated herein by reference;
x. Prodrugs, e.g., physiologically hydrolysable and acceptable esters of esters of VIP;
   in free or pharmaceutically acceptable salt form. Human VIP is preferred. Human VIP may be produced, e.g. recombinantly or synthetically, preferably recombinantly, and may be provided, e.g., in the form of the amide.

Administration routes of the T3/VIP nanoparticle include, but are not limited to intravenous, intra-arterial, intracardiac, subcutaneous, intramuscular, orally, intrapulmonary (e.g., by inhalation), intradermal, topically or rectally. The formulation may be for immediate release, e.g., via intravenous, intra-arterial, or intracardiac injection, or may be in the form of a sustained release depot formulation, e.g., a depot comprising a biodegradable polymer comprising the T3/VIP nanoparticles of the invention, for example for subcutaneous or intramuscular injection, resulting in release of T3 and/or VIP over a period of days or weeks.

In a further aspect, administration routes can include interosseous applications or intraosseous applications. In one aspect, it is contemplated that T3/VIP nanoparticle formulations as described herein may be administered directly into the bone marrow of patient. In one aspect, for example, where the patient's blood flow through the veins is diminished or decreased. Accordingly, in one aspect, interosseous or intraosseous applications of the device described herein would be advantageous for the delivery of the T3/VIP nanoparticle formulation. In another aspect, it is contemplated that interosseous or intraosseous administration could be used in order to treat certain diseases or disorders which primarily affect bone.

The methods allow for delivery of T3 and/or VIP in a few minutes and provide sustained elevated serum concentration of T3 and/or VIP over time.

In one example of the method, the T3/VIP-nanoparticles may be lyophilized. The nanoparticles are stable with long shelf life. The T3/VIP-nanoparticles may be dispensable in physiological saline. The formulations may for example have a pH of 7.0-7.8, e.g., 7.4.

In one embodiment the T3/VIP nanoparticles can be used in a drug-eluting metal or bioresorbable stent, e.g., for patients having had or at risk of acute myocardial infarction, e.g., for insertion in the coronary artery.

In a further embodiment, the T3/VIP-eluting stents are also useful for patients with a history of stroke or transient ischemic attacks or patients otherwise at risk of stroke, e.g., for placement in the carotid artery, or for patients having pulmonary hypertension, e.g. for placement in the pulmonary artery.

In one embodiment, administration is by a pump activated by a signal, which releases the nanoparticles into the bloodstream. In one embodiment the signal is generated when pulmonary arterial pressure rises above a given level, e.g., greater than 30, for example, greater than 40 mmHg, as measured by an electronic pressure transducer linked to a cannula in the pulmonary artery. In another embodiment, the signal is generated when oxygen levels in the blood drop below a certain level, e.g., % SpO2 below 90, e.g., below 85 as measured by a pulse oximeter.

In one embodiment, the particles provide a sustained release which allows the T3/VIP nanoparticle to affect gene expression.

The T3/VIP nanoparticles of the invention (e.g., the nanoparticle of any of a.)-x.)) may be administered in conjunction with, or adjunctive to, the normal standard of care for pulmonary hypertension or cardiac insufficiency or other cardiovascular or neurological disorders, for example in conjunction with one or more of:
y. Drugs selected from the group consisting of endothelin receptor antagonists (e.g., bosentan, sitaxentan, ambrisentan), phosphodiesterase type 5 inhibitors (e.g., sildenafil, tadalafil), prostacyclin derivatives (e.g., epoprostenol, treprostenil, iloprost, beroprost), and soluble guanylate cyclase (sGC) activators (e.g., cinaciguat and riociguat).
z. Diuretics, e.g., hydrochlorothiazide
aa. Anticoagulants, e.g., Coumadin, aspirin
bb. Calcium channel blockers, e.g., amlodipine
cc. Beta-blockers, e.g. metoprolol
dd. ACE inhibitors, e.g. captopril, enalapril
ee. Nitrates, e.g. nitroglycerin
ff. Inhaled beta-agonists, corticosteroids, and/or anticholinergics
gg. Other antihypertensives

Various methods of synthesizing T3/VIP-nanoparticles are provided. For example, a single emulsion process may produce chitosan-PLGA nanoparticles encapsulating and/or immobilizing T3 and VIP. In yet another example, a process involving gelation/conjugation of preformed biodegradable polymers produces 1) chitosan nanoparticles encapsulating and/or immobilizing T3 and VIP with and without glutaraldehyde as a cross-linker; or 2) chitosan-PLGA nanoparticles encapsulating and/or immobilizing T3 and VIP. Other cross-linkers may be used.

In yet another example, a process involving chemical bonding of T3 and VIP on the surface of chitosan-PLGA nanoparticles produces 1) chitosan-PLGA nanoparticles immobilizing T3 and VIP or 2) chitosan-PLGA nanoparticles immobilizing T3 and VIP and additionally including chitosan-PLGA nanoparticles encapsulating VIP.

For example, in one embodiment, PLGA and T3 and VIP are first immersed in a 1% PVA solution and chitosan. They are then stirred and sonicated. Then a dialysis step is performed. After a dialysis step occurs, PLGA-chitosan nanoparticles encapsulating T3 and VIP are produced. Then, in the final step, the nanoparticles may then have a chitosan layer cross-linked with glutaraldehyde. Other cross-linkers may be used.

An entrapment efficiency may also be measured. The entrapment efficiency may be calculated to be the total amount of VIP in the nanoparticles/initial concentration of VIP added to make the formulation×100.

The methods using the T3/VIP nanoparticles of the present invention (e.g., the nanoparticle of any of a.)-y.)) may be use to treat acute cardiac insufficiency. Examples of cardiac conditions that may be treated include cardiac arrest, cardiogenic shock, and acute heart failure. Additionally, the T3/VIP nanoparticles may be used to treat chronic cardiac conditions, e.g., right and left sided heart failure, right-sided heart failure that is the result of pulmonary hypertension. The T3/VIP nanoparticles may also me used to treat intra-abdominal pressure and/or intra-abdominal hypertension.

For example, while not bound by theory, for cardiac the delivery of T3/VIP-nanoparticles may act rapidly and directly in order to increase the effective mechanical contraction of the heart, decrease systemic vascular resistance, and increase heart rate. Additionally, while not bound by any theory, for chronic cardiac conditions it is believed that VIP may be able to decrease pressure, while T3 may be able lower systemic vascular resistance. T3 may enhance cardiac contractility, in addition to lowering systemic vascular resistance. Contemporaneously, VIP may decrease pressure associated with pulmonary hypertension while also promoting vascular and/or cardiac remodeling. The vascular and cardiac remodeling properties of VIP could be especially useful following damage from myocardial infarction, for example.

In another embodiment, the present invention provides for methods of treating intra-abdominal pressure, intra-abdominal hypertension, and cardiorenal failure. While not bound by theory, it is believed that cardiorenal failure may be caused by the compression of veins and/or right ventricular heart failure. The administration of a T3/VIP nanoparticle may serve to dilate compressed veins and act locally to ameliorate right ventricular heart failure.

In another embodiment, the present invention provides for a method of treating autoimmune disorders, e.g. scleroderma, lupus, as they relate to pulmonary hypertension and cardiac conditions, e.g. heart failure, comprising administering an effective amount of a T3/VIP nanoparticle formulation to a patient in need thereof.

In another embodiment, the present invention contemplates the use of a T3/VIP nanoparticulate formulation to treat organs used in transplantation. Without being bound by theory, the T3/VIP nanoparticle may assist in increasing the donor organ longevity, possibly by decreasing the incidence of acute injury. Again, without being bound by theory, the T3/VIP nanoparticle may also assist by decreasing the incidence of chronic or long-term injury that may occur several months, or years, after the organ has been transplanted into a patient. It is believed that the T3/VIP nanoparticle could assist in preventing injury by preventing the incidence of reperfusion injury in a donor organ.

In one embodiment, the T3/VIP of the present invention particles provide a sustained release which allows the T3 to affect gene expression. In another embodiment, the T3 is covalently linked to the bioabsorbable polymer, which reduces the genomic effect and enhances the effect on the integrin receptor, and the VIP is encapsulated by chitosan.

The T3/VIP nanoparticles of the invention may be administered in conjunction with, or adjunctive to, the normal standard of care for cardiac arrest, e.g., cardiopulmonary resuscitation, defibrillation, and epinephrine. They may be administered shortly after the cardiac arrest, and optionally later, e.g., 8-24 hours later, to preserve cardiac function.

Various methods of synthesizing T3/VIP-nanoparticles are provided. For example, a single emulsion process may produce chitosan-PLGA nanoparticles encapsulating T3 and VIP. In yet another example, a process involving gelation/conjugation of preformed biodegradable polymers produces 1) chitosan nanoparticles encapsulating T3 and/or VIP with and without glutaraldehyde as a cross-linker; or 2) chitosan-PLGA nanoparticles encapsulating T3 and/or VIP. Other cross-linkers may be used.

In yet another example, a process involving chemical bonding of T3 and/or VIP on the surface of chitosan-PLGA nanoparticles produces 1) chitosan-PLGA nanoparticles immobilizing T3 or 2) chitosan-PLGA nanoparticles immobilizing T3 and/or VIP and additionally including chitosan-PLGA nanoparticles encapsulating T3 and/or encapsulating VIP.

1. A formulation comprising T3/VIP nanoparticles, wherein the T3/VIP nanoparticle comprises T3 and VIP encapsulated or immobilized on a bioabsorbable polymer.

2. The formulation of claim 1 wherein the polymer comprises chitosan.

3. The formulation of claim 1 wherein the polymer comprises poly(lactic-co-glycolic acid) (PLGA).

4. The formulation of claim 1 wherein the polymer comprises chitosan crosslinked using glutaraldehyde.

5. The formulation of claim 1 wherein the polymer comprises chitosan linked to bile acids.

6. The formulation of claim 1 wherein the polymer comprises chitosan linked to PLGA.

7. The formulation of claim 1 wherein the nanoparticles have an average diameter of 50-1000 nm.

8. The formulation of claim 1 wherein the nanoparticles have a zeta potential of 10-100 mV.

9. The formulation of claim 1 wherein the nanoparticle comprises a second pharmacologically active ingredient.

10. The formulation of claim 1 comprising T3 and VIP in a nanoparticle, wherein T3 is covalently bound to the polymer and VIP is not covalently bound to the polymer.

11. The formulation of claim 1 comprising T3 and VIP in a nanoparticle, wherein T3 is not covalently bound to the polymer and VIP is covalently bound to the polymer.

12. The formulation of claim 1 comprising both T3 and VIP in a nanoparticle, wherein both T3 and VIP are covalently bound to the polymer.

13. The formulation of claim 1 comprising both T3 and VIP in a nanoparticle, wherein neither T3 nor VIP are covalently bound to the polymer.

14. The formulation of claim 1 wherein the nanoparticle comprises T3, VIP, PLGA, and chitosan, wherein VIP is encapsulated inside said chitosan and is released immediately and wherein T3 is encapsulated by PLGA and is released subsequent to the release of VIP.

15. A composition of claim 1, wherein T3 and VIP are covalently linked to chitosan.

16. The composition of claim 15 wherein the linkage is between the amino groups on the chitosan and the phenolic hydroxy on the VIP and/or T3.

17. A method of making a T3/VIP nanoparticle, comprising:
providing PLGA and T3 and/or VIP;
immersing the PLGA and T3 and/or VIP in a 1% solution including chitosan;
stirring and sonicating; and
performing a dialysis step, to yield the T3/VIP nanoparticle.

18. The method of claim 17, further comprising a step of crosslinking a chitosan layer formed in the T3/VIP-nanoparticle, with a cross-linker.

19. The method of claim 18, wherein the step of crosslinking utilizes glutaraldehyde as the cross-linker.

20. A T3/VIP nanoparticle obtained or obtainable by the methods of claim 16.

21. Method of making a T3/VIP nanoparticle of claim 1, comprising covalently linking either T3 and/or VIP to a bioabsorbable polymer.

22. Method of claim 21 comprising reacting either T3 and/or VIP with a bioabsorbable polymer having amino moieties.

23. Method of claim 21 wherein the bioabsorbable polymer comprises chitosan, wherein the chitosan is optionally crosslinked, and/or linked to bile acids and/or linked to poly(lactic-co-glycolic acid).

24. Method of claim 20 comprising covalently linking either T3 and/or VIP to the surface of a nanoparticle.

25. T3/VIP nanoparticle obtained or obtainable by the method of claim 21.

26. A method for treating pulmonary hypertension, comprising administering an effective amount of a T3/VIP nanoparticle formulation of claim 1 to a patient in need thereof.

27. The method of claim 26, wherein the pulmonary hypertension is pulmonary arterial hypertension.

28. The method of claim 26 further comprising administering a drug selected from the group consisting of endothelin receptor antagonists, phosphodiesterase type 5 inhibitors, prostacyclin derivatives, and soluble guanylate cyclase (sGC) activators.

29. A method of treating cardiac insufficiency comprising administering a T3/VIP nanoparticle formulation of claim 1.

30. (canceled)

31. (canceled)

32. (canceled)

33. (canceled)

34. (canceled)

35. (canceled)

36. A method for treating cardiac insufficiency, comprising administering a therapeutically effective amount of a T3/VIP nanoparticle formulation of claim 1 to a patient in need thereof.

37. The method of claim 36, wherein said cardiac insufficiency is heart failure, angina, or acute myocardial infarction.

38.-43. (canceled)
